(19) United States
(12) Patent Application Publication
(10) Pub. No.: US 2018/0030475 A1
(43) Pub. Date: Feb. 1, 2018

(54) METHODS FOR PRODUCING MODIFIED RED BLOOD CELL COMPOSITIONS, COMPOSITIONS AND USES THEREOF

(71) Applicant: Jeffrey Thomas LOH, Honolulu, HI (US)

(72) Inventor: Jeffrey Thomas LOH, Honolulu, HI (US)

(21) Appl. No.: 15/553,522

(22) PCT Filed: Mar. 29, 2016
(86) PCT No.: PCT/US16/24805

§ 371 Date: Aug. 24, 2017

Related U.S. Application Data
(60) Provisional application No. 62/139,931, filed on Mar. 30, 2015.

(51) Int. Cl. C12N 15/85; C12N 15/63

(52) U.S. Cl. CPC C12N 15/85; C12N 15/63

(57) ABSTRACT

The present invention encompasses methods for generating JAK2-modified cultured red blood cells (modified cRBCs) expressing a mutant Janus kinase 2 peptide, JAK2-modified cRBCs as a composition of matter, and methods for using the generated JAK2-modified cRBCs.

### CROSS-REFERENCE TO RELATED APPLICATIONS

The present application is a U.S. 371 National Phase filing of International PCT Application No. PCT/US2016/024805, filed Mar. 29, 2016, which claims priority to U.S. Provisional Application No. 62/139,931, filed Mar. 30, 2015, which is herein incorporated by reference.

### FIELD OF THE INVENTION

The field of the invention encompasses methods for generating JAK2-modified cultured red blood cells (modified cRBCs) expressing a mutant Janus kinase 2 peptide, JAK2-modified cRBCs as a composition of matter, and methods for using the generated JAK2- modified cRBCs.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Red blood cell (RBC) transfusions, developed in the 1930s, are the most commonly used cell-based therapy in use today. In 2009, more than 17.3 million units of RBCs were collected in the United States. Currently, RBC transfusions are used extensively in emergency medicine, serve as an essential component of surgical procedures and chemotherapy, and are one of the major treatment options for individuals with hereditary anemias, including β-thalasseimia major and sickle cell disease. In the Western world, a volunteer-based collection system covers most transfusion needs; however, the existing transfusion system is expensive to maintain and is vulnerable to major disruptions that could be caused by the emergence of novel pathogens, including hazardous viruses and prions.

Thus, there is a need in the art for a universal, limitless source of red blood cells, such as cultured red blood cells. The present invention provides methods and compositions that address this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description, including those aspects illustrated in the accompanying drawings and defined in the appended claims.

In some embodiments, the present invention provides a method of producing mature Janus kinase 2 V617F (mutant JAK2) modified cultured red blood cells comprising: providing human pluripotent stem cells or human blood cells; transforming the human pluripotent stem cells or human blood cells with an expression vector expressing mutant JAK2 or creating a mutation in an endogenous JAK2 locus in the human pluripotent stem cells or human blood cells to produce modified cells; culturing the modified cells; inducing enucleation of the modified cells; and isolating the enucleated modified cells.

In some aspects of this embodiment, the human pluripotent stem cells or human blood cells are human embryonic stem cells, human embryonal carcinoma cells, human embryonic germ cells, human multipotent germline cells, human mesodermal stem cells, human mesenchymal stem cells, human induced pluripotent stem cells, or human erythroid progenitor cells. In preferred aspects, the human pluripotent stem cells or human blood cells are human embryonic stem cells, human induced pluripotent stem cells, or erythroid progenitor cells. In more preferred aspects of this embodiment, the human pluripotent stem cells or human blood cells are ABO type O and RhD negative.

In some aspects, the human pluripotent stem cells or human blood cells are transformed with a mutant JAK2 expression vector; in other aspects, the human pluripotent stem cells or human blood cells comprise a human synthetic chromosome expressing mutant JAK2; and yet other aspects, the endogenous JAK2 locus of the human pluripotent stem cells or human blood cells is replaced with mutant JAK2 via homologous recombination. In preferred aspects, the mutant JAK2 is under control of an inducible promoter. In yet other aspects, human pluripotent stem cells or human blood cells are taken from an individual with, e.g., polycythemia vera, such that the human pluripotent stem cells or human blood cells naturally comprise an endogenous mutant JAK2 locus.

In some aspects, the modified cells are cultured in the presence of one or more of human insulin-like growth factor-II, human vascular endothelial growth factor, human stem cell factor, human erythropoietin or dexamethasone, and in some aspects, the modified cells are cultured first in human insulin-like growth factor-II and human vascular endothelial growth factor, followed by culture in human stem cell factor, human erythropoietin and dexamethasone. Also in some aspects, the modified cells are cultured in the presence of feeder cells, and in some aspects, the feeder cells are OP9 cells, MEF cells, SNL76/7 cells, PA6 cells, NIH3T3 cells, M15 cells, or 10T1/2 cells.

In some aspects of the method embodiment described above, enucleation is induced by culturing the modified cells with a human stromal cell line, where in other aspects, enucleation is induced by culture in one or more of human stem cell factor, human erythropoietin, human interleukin 3, human vascular endothelial growth factor or human insulin-like growth factor-II.

Other embodiments of the present invention provide mature, enucleated cultured red blood cells produced by the methods described herein, and other embodiments provide a method of treating a human patient comprising transfusing the patient with the mature, enucleated cultured red blood cells produced by the methods of the present invention. In preferred embodiments, the mature, enucleated cultured red blood cells produced by the methods of the present invention are ABO type O and RhD negative.

Yet other embodiments of the present invention provide a method of producing an immortalized modified mutant Janus kinase 2 V617F (mutant JAK2) erythroid progenitor cell line comprising: providing human pluripotent stem cells or human blood cells; transforming the human pluripotent stem cells or human blood cells with an expression vector expressing mutant JAK2 or creating the Janus kinase 2 V617F mutation in an endogenous JAK2 locus in the human pluripotent stem cells or human blood cells to produce modified cells; and culturing the modified cells in nondifferentiating blood stem/blood progenitor cell culture medium. In some aspects of this embodiment, mature, enucleated Janus kinase 2 V617F (mutant JAK2) modified cultured red blood cells are produced from the immortalized modified mutant Janus kinase 2 V617F (mutant JAK2) erythroid progenitor cell line by inducing enucleation of the modified cells; and isolating the enucleated modified cells.

These and other aspects and uses of the invention will be described in the detailed description.

### BRIEF DESCRIPTION OF THE FIGURES

FIG. 1 is a simplified flow chart of method steps for creating JAK2-modified cultured red blood cells (modified cRBCs), and for using the modified cRBCs.

### DETAILED DESCRIPTION OF THE INVENTION

The methods described herein may employ, unless otherwise indicated, conventional techniques and descriptions of molecular biology (including recombinant techniques), cell biology, biochemistry, and cellular engineering technology, all of which are within the skill of those who practice in the art. Such conventional techniques include oligonucleotide synthesis, hybridization and ligation of oligonucleotides, transformation and transduction of cells, engineering of recombination systems, differentiation of cells and maintenance in cell culture, and human therapy. Specific illustrations of suitable techniques can be had by reference to the examples herein. However, equivalent conventional procedures can, of course, also be used. Such conventional techniques and descriptions can be found in standard laboratory manuals such as Genome Analysis: A Laboratory Manual Series (Vols. I-IV) (Green, et al., eds., 1999); Genetic Variation: A Laboratory Manual (Weiner, et al., eds., 2007); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Protein Methods (Bollag et al., John Wiley & Sons 1996); Nonviral Vectors for Gene Therapy (Wagner et al. eds., Academic Press 1999); Viral Vectors (Kaplift & Loewy, eds., Academic Press 1995); Immunology Methods Manual (Lefkovits ed., Academic Press 1997); Gene Therapy Techniques, Applications and Regulations From Laboratory to Clinic (Meager, ed., John Wiley & Sons 1999); M. Giacca, Gene Therapy (Springer 2010); Gene Therapy Protocols (LeDoux, ed., Springer 2008); Cell and Tissue Culture: Laboratory Procedures in Biotechnology (Doyle & Griffiths, eds., John Wiley & Sons 1998); Essential Stem Cell Methods, (Lanza and Klimanskaya, eds., Academic Press 2011); Stem Cell Therapies: Opportunities for Ensuring the Quality and Safety of Clinical Offerings: Summary of a Joint Workshop (Board on Health Sciences Policy, National Academies Press 2014); Essentials of Stem Cell Biology, Third Ed., (Lanza and Atala, eds., Academic Press 2013); and Handbook of Stem Cells, (Atala and Lanza, eds., Academic Press 2012), all of which are herein incorporated by reference in their entirety for all purposes. Before the present compositions, research tools and methods are described, it is to be understood that this invention is not limited to the specific methods, compositions, targets and uses described, as such may, of course, vary. It is also to be understood that the terminology used herein is for the purpose of describing particular aspects only and is not intended to limit the scope of the present invention, which will be limited only by the appended claims.

Note that as used in the present specification and in the appended claims, the singular forms “a,” “and,” and “the” include plural referents unless the context clearly dictates otherwise. Thus, for example, reference to “a composition” refers to one or mixtures of compositions, and reference to “an assay” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned are incorporated herein by reference for the purpose of describing and disclosing devices, formulations and methodologies which might be used in connection with the present invention.

Where a range of values is provided, it is understood that each intervening value between the upper and lower limit of that range and any other stated or intervening value in that stated range is encompassed within the invention. The upper and lower limits of these smaller ranges may independently be included in the smaller ranges, subject to any specifically excluded limit in the stated range. Where the stated range includes both of the limits, ranges excluding only one of those included limits are also included in the invention.

In the following description, numerous specific details are set forth to provide a more thorough understanding of the present invention. However, it will be apparent to one of ordinary skill in the art upon reading the specification that the present invention may be practiced without one or more of these specific details. In other instances, features and procedures well known to those skilled in the art have not been described in order to avoid obscuring the invention.

### Definitions

Unless expressly stated, the terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

As used herein “blood stem cells” means stem cells having no differentiation potential to cells other than blood cells but having a differentiation potential to various types of blood cells. “Blood stem cells” are also called “hematopoietic stem cells.” Blood stem cells are known to be abundantly included in cell populations separated and collected from certain tissues, such as umbilical cord blood, peripheral blood, bone marrow, or fetal liver by, e.g., flow cytometry or the like using an antibody that binds specifically to a cell surface antigen such as, e.g., CD34 on hematopoietic stem cells. The blood stem cells of the present invention can be prepared by inducing differentiation of human pluripotent stem cells. “Human pluripotent stem cells” as used herein may be any human cells that renew and can be induced to differentiate into blood stem cells. Examples of human pluripotent stem cells include human embryonic stem cells (ES cells), human embryonal carcinoma cells (EC cells), human embryonic germ cells (EG cells), human multipotent germline stem cells (mGS cells), human mesodermal stem cells, human mesenchymal stem cells and the like. In addition, an example of human pluripotent stem cells includes cells artificially prepared in such a manner as to have differentiation pluripotency, such as induced pluripotent stem cells (iPSCs). An “erythroid progenitor cell” means a cell having a differentiation potential to only mature red blood cells and to no other cell. An “erythroid progenitor cell line” means immortalized erythroid progenitor cells, which can be maintained in culture through many passings.

A “blood stem/progenitor cell differentiation induction culture protocol” or “blood stem/progenitor cell differentiation induction culture medium” refers to protocols or cell culture media that are useful for inducing differentiation of human pluripotent stem cells to human blood stem cells and further to erythroid cells. For example, human pluripotent stem cells may be co-cultured with feeder cells in the presence of human insulin-like growth factor-II (IGF-II) and human vascular endothelial growth factor (VEGF) to induce differentiation into blood cells including blood stem cells. Then, by co-culturing with feeder cells in the presence of human stem cell factor (SCF), human erythropoietin (EPO) and dexamethasone (DEX) in place of IGF-II and VEGF, differentiation into a cell population including erythroid cells can be induced. Thus, in such a case, a “blood stem/progenitor cell differentiation induction culture medium” comprises cell media supplemented with IGF-II and VEGF, then SCF, EPO and DEX.

A “coding sequence” or a sequence that “encodes” a peptide is a nucleic acid molecule that is transcribed (in the case of DNA) and translated (in the case of mRNA) into a polypeptide in vivo when placed under the control of appropriate control sequences. The boundaries of the coding sequence typically are determined by a start codon at the 5′ (amino) terminus and a translation stop codon at the 3′ (carboxy) terminus.

The term DNA “control sequences” refers collectively to promoter sequences, polyadenylation signals, transcription termination sequences, upstream regulatory domains, origins of replication, internal ribosome entry sites, enhancers, and the like, which collectively provide for the replication, transcription and translation of a coding sequence in a recipient cell. Not all of these types of control sequences need to be present so long as a selected coding sequence is capable of being replicated, transcribed and translated in an appropriate host cell.

The term “in vitro” refers to events that occur in an artificial environment, e.g., in a test tube or reaction vessel, in cell culture, etc., rather than within an organism.

The terms “heterologous DNA” or “foreign DNA” (or “heterologous RNA” or “foreign RNA”) are used interchangeably and refer to DNA or RNA that does not occur naturally as part of the genome in which it is present, or is found in a location or locations and/or in amounts in a genome or cell that differ from that in which it occurs in nature. Examples of heterologous DNA include, but are not limited to, DNA that encodes a gene product or gene product(s) of interest. Other examples of heterologous DNA include, but are not limited to, DNA that encodes traceable marker proteins as well as regulatory DNA sequences.

“Operably linked” refers to an arrangement of elements where the components so described are configured so as to perform their usual function. Thus, control sequences operably linked to a coding sequence are capable of effecting the expression of the coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome), and may still have interactions resulting in altered regulation.

A “promoter” or “promoter sequence” is a DNA regulatory region capable of binding RNA polymerase in a cell and initiating transcription of a polynucleotide or polypeptide coding sequence such as messenger RNA, ribosomal RNAs, small nuclear or nucleolar RNAs or any kind of RNA transcribed by any class of any RNA polymerase I, II or III.

As used herein the term “selectable marker” refers to a gene introduced into a cell, particularly in the context of this invention into cells in culture that confers a trait suitable for artificial selection. General use selectable markers are well-known to those of ordinary skill in the art. In preferred embodiments, selectable markers for use to modify and/or propagate cRBCs should be non-immunogenic in the human and include, but are not limited to: human nerve growth factor receptor (detected with a monoclonal antibody (MAb), such as described in U.S. Pat. No. 6,365,373); truncated human growth factor receptor (detected with a MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34⁺ cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by MAb-FITC); Methylguanine-DNA methyltransferase (MGMT; selectable by carmustine); and Cytidine deaminase (CD; selectable by Ara-C). Drug selectable markers such as puromycin, hygromycin, blasticidin, G418, tetracycline may also be employed. In addition, using FACs sorting, any fluorescent marker gene may be used for positive selection, as may chemiluminescent markers (e.g. Halotags), and the like.

The terms “subject”, “individual” or “patient” may be used interchangeably herein and refer to a mammal, and in preferred embodiments, a human.

As used herein, the terms “treat,” “treatment,” “treating,” and “amelioration” refer to therapeutic treatments, wherein the object is to reverse, alleviate, ameliorate, inhibit, slow down and/or stop the progression or severity of a condition associated with a disease or disorder. The terms include reducing or alleviating at least one adverse effect or symptom of a condition, disease or disorder associated with a deficiency in the number or defect in the quality of at least one blood cell type, such as platelets. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The terms “treat,” “treatment,” “treating,” and “amelioration” in reference to a disease also include providing relief from the symptoms or side-effects of the disease (including palliative treatment).

A “vector” is a replicon, such as plasmid, phage, viral construct, cosmid, bacterial artificial chromosome, derived artificial chromosome or yeast artificial chromosome to which another heterologous DNA segment may be inserted. In some instances a vector may be a chromosome such as in the case of an arm exchange from one endogenous chromosome engineered to comprise a recombination site to a synthetic chromosome. Vectors are used to transduce and express a DNA segment, such as a mutated Janus kinase 2 gene (JAK2) in a cell.

### The Invention

The in vitro production of cultured red blood cells (cRBCs) has recently emerged as a potential long-term alternative to the current donor-based RBC procurement system. The current donor-based system is expensive to maintain, is vulnerable to major disruption, and does not adequately serve the needs of chronically-transfused patients who often require RBCs expressing rare blood groups. Production of cRBCs from stem cells holds the promise of changing the paradigm for transfusion medicine and overcoming dependence on the existing RBC supply system. The use of terminally differentiated cells that no longer have the capability of proliferating allows clinical applications of human pluripotent and blood stem cells without the associated risk of tumorigenicity, as RBCs lack nuclei following terminal differentiation and are highly unlikely to exhibit tumorigenicity in vivo. Thus, even if the original stem cells or their derivatives possessed abnormal karyotypes or genetic mutations, these cells might be useful for clinical applications provided that such precursors can produce enucleated RBCs. Another advantage is that ex vivo-generated RBCs—like donor-based RBCs—need to be compatible with recipient ABO and RhD antigens; however, establishment of an immortalized human erythroid cell line lacking the genes to produce A, B, and RhD antigens would produce O/RhD- RBCs, which in theory are transfusable into all individuals. Moreover, it is possible to further engineer such an immortalized human erythroid cell line to negate or disable other cell surface antigens to avoid immune reactions in chronically-transfused patients.

The present invention encompasses compositions and methods for producing Janus kinase 2-modified cultured red blood cells (JAK-2 modified RBCs). Janus kinase 2 (JAK2) is a non-receptor tyrosine kinase that has been implicated in signaling by members of the type II cytokine receptor family (e.g., interferon receptors), the GM-CSF receptor family, the gp 130 receptor family, and single chain receptors. JAK2 signaling appears to be activated downstream from the prolactin receptor. The distinguishing feature between Janus kinase 2 and other JAK kinases is the lack of Src homology binding domains and the presence of up to seven JAK homology domains. Mutations in the Janus kinase 2 gene (herein “JAK2”, corresponding to Entrez Gene ID:3717 and Uniprot O60674) have been implicated in polycythemia vera (a disorder in which the bone marrow makes too many red blood cells), essential thrombocythemia, myelofibrosis as well as other myeloproliferative disorders. The specific mutation, a change of valine to phenylalanine at the 617 position (V617F, herein “mutated JAK2” or “mutant JAK2” for the peptide or “mutated JAK2” or “mutant JAK2” for the gene that codes for the peptide with the change of valine to phenylalanine at the 617 position or codes for a conservative substitution therefor) appears to render hematopoietic cells more sensitive to growth factors such as erythropoietin and thrombopoietin.

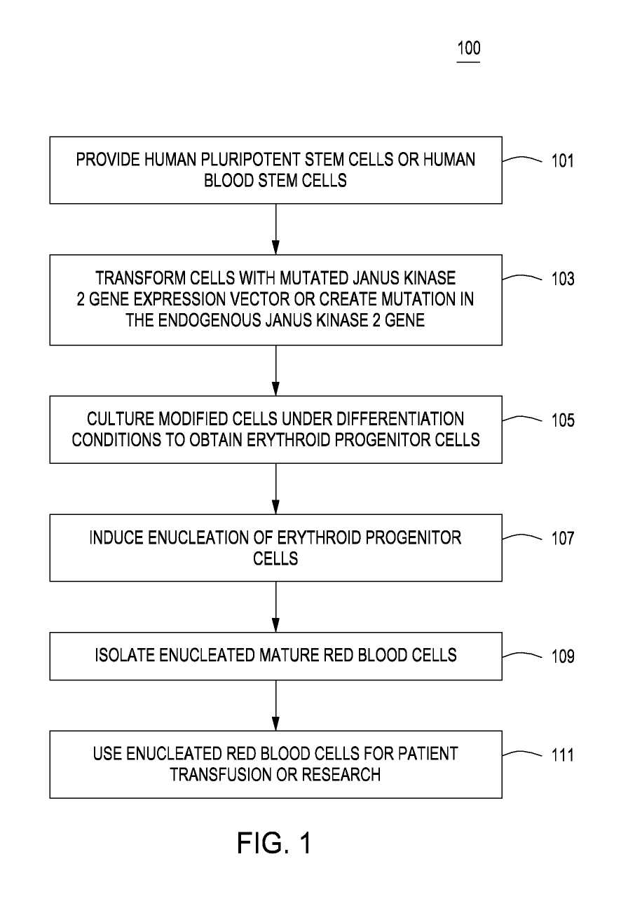

FIG. 1 is a simplified flow chart of a method 100 for creating mutant JAK2-modified cultured red blood cells (modified cRBCs). First, human pluripotent stem cells or blood stem cells are provided in step 101. In step 103, the human pluripotent stem cells or blood stem cells are transformed with mutated JAK2 or a mutation is created in endogenous JAK2 to create modified human pluripotent stem cells or blood stem cells. In step 105, the modified human pluripotent stem cells or blood stem cells are maintained in culture in an undifferentiated state and later differentiated into erythroid progenitor cells, or are differentiated into erythroid progenitor cells and then the erythroid progenitor cells are maintained in culture. Note that it is possible to reverse steps 103 and 105; that is, the human pluripotent stem cells or blood stem cells may be differentiated into erythroid progenitor cells before a mutation in JAK2 is created in the cells. Alternatively, it is possible to culture human pluripotent stem cells, blood stem cells or erythroid progenitor cells from an individual who has a mutation in JAK2 naturally as a basis for the JAK2-modified cRBCs; that is, blood stem cells that naturally comprise an endogenous JAK2 mutation—such as blood stem cells taken from an individual with polycythemia vera—may be used in lieu of engineered cells. Either way, once JAK2-modified erythroid progenitor cells are obtained, the cells can be maintained in culture indefinitely, or enucleation of the erythroid progenitor cells is induced (step 107), thus producing enucleated (mature) red blood cells at step 109. The mature mutant JAK2-modified cRBCs can then be used for patient transfusion, or in other uses in step 111. The details of each step outlined in the simplified flow chart are described below.

### Cells

Human pluripotent stem cells, human blood stem cells or human erythroid progenitor cells can be used in the present invention, depending on the availability of each type of cell, and protocols that have been developed to differentiate stem cells and immortalize cell lines. Human RBCs have a limited life span and are the progeny of immortal self-renewing hematopoietic stems cells. Approximately 1% of circulating RBCs are eliminated from the body every day; thus, maintenance of the average adult RBC mass of 2.5×10¹³ requires the daily production of more than 200 billion RBCs. Differentiation of hematopoietic stem cells into RBCs involves the generation of a series of progenitors with increasingly restricted differentiation potential. That is, RBCs differentiate sequentially into common myeloid progenitors and megakaryocyte-erythroid progenitors, and then into unipotent progenitors restricted to the erythroid lineage (erythroid progenitor cells).

One source of human stem and progenitor cells is circulating stem and progenitor cells. Laboratory-scale methods to produce cRBCs from circulating stem and progenitor cells have been developed. The best methods currently available allow for the stem and progenitor cells found in 1 unit of cord blood to be expanded into more than 500 units of RBCs (see, e.g., Fujimi, et al., Int. J. Hematology, 87:339-50 (2005); Legerbaure, et al., Blood, 105:85-94 (2005); Giarratana, et al., Nat. Biotechnology, 23:69-74 (2005); and Timmins, et al., Tissue Eng. Part C Methods, 1:103-21 (2000)). The main drawback of the use of circulating human stem and progenitor cells is that currently there is a limited proliferation potential.

Another source of human stem and progenitor cells is the generation of immortalized progenitors that can be grown in large amounts in simple medium yet the progenitor cells retain their capacity to differentiate into RBCs upon induction. To achieve cellular immortalization, proliferation of the cells must be stimulated, while terminal differentiation must be inhibited. To date, several factors have been identified that control these processes, including GATA-1, which promotes erythroid development; PU.1, which binds to GATA-1 and inhibits erythroid terminal differentiation; anti-apoptotic proteins BCL-2 and BCL-XL; c-Kit, which promotes erythroid progenitor differentiation; as well as c-Myc, a well-known immortalizing transcription factor.

A preferred source of cells for RBC production is pluripotent stem cells such as human embryonic stem cells (ESCs) and induced pluripotent stem cells (iPSCs). The main advantage of these cells is that they are immortal, karyotypically stable and can be reproducibly generated from any individual using a variety of well-developed methods (see, e.g., Okita, et al., Philosophical Transaction of the Royal Society of London Biological Sciences, 366:2198-207 (2011)). The capacity of human pluripotent cells to differentiate into the erythroid lineage has been demonstrated, and methods that do not use any xenobiotic components have been developed (see, e.g., Mazurier, et al., Curr. Opin. Hematol., 18:249-53 (2011)).

Alternatively, it is possible to culture human pluripotent stem cells, blood stem cells or erythroid progenitor cells from an individual who has a mutation in JAK2 naturally as a basis for the JAK2-modified cRBCs; that is, blood stem cells that naturally comprise an endogenous JAK2 mutation—such as blood stem cells taken from an individual with polycythemia vera—may be used in lieu of engineered cells.

### Mutant JAK2 Expression in Hematopoietic Stem Cells

Methods to introduce a mutated JAK2 or to replace endogenous JAK2 with mutated JAK2 are generally known to those in the art. For example, a viral or non-viral vector engineered to express mutated JAK2 can be introduced into the erythroid progenitor cells, blood stem cells or human pluripotent stem cells of choice. Alternatively, a blood stem cell line, human pluripotent stem cell line or erythroid progenitor cell line can be engineered to produce a human synthetic chromosome that is engineered to express mutated JAK2. In yet another alternative, endogenous JAK2 in a erythroid progenitor cell, blood stem cell, or human pluripotent stem cell can be replaced via homologous recombination systems with mutated JAK2.

In the first alternative, the choice of vector to be used in delivery of mutated JAK2 to the cell of choice will depend upon a variety of factors such as the type of cell in which propagation is desired. Certain vectors are useful for amplifying and making large amounts of a desired DNA sequence such as in this case, mutated JAK2, while other vectors are suitable for expression in cells in culture. The choice of an appropriate vector is well within the skill of those in the art, and many vectors are available commercially. To prepare the constructs, a mutated JAK2 polynucleotide is inserted into a vector, typically by means of ligation into a cleaved restriction enzyme site in the vector.

Exemplary vectors that may be used include but are not limited to those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Additional vectors include bacterial artificial chromosomes (BACs) based on a functional fertility plasmid (F-plasmid), yeast artificial chromosomes (YACs), and P1-derived artificial chromosomes, DNA constructs derived from the DNA of P1 bacteriophage (PACS). Alternatively and preferably, recombinant virus vectors may be engineered, including but not limited to those derived from viruses such as herpes virus, retroviruses, vaccinia virus, poxviruses, adenoviruses, lentiviruses, adeno-associated viruses or bovine papilloma virus.

Whichever vector is chosen, typically an expression cassette expressing mutated JAK2 is employed. An expression vector provides transcriptional and translational regulatory sequences, and may provide for inducible or constitutive expression, where the coding region is operably linked under the transcriptional control of the transcriptional initiation region and a transcriptional and translational termination region. These control regions may be native to JAK2 or may be derived from exogenous sources, including species-specific endogenous promoters. In general, the transcriptional and translational regulatory sequences may include, but are not limited to, promoter sequences, ribosomal binding sites, transcriptional start and stop sequences, translational start and stop sequences, and enhancer or activator sequences. In addition to constitutive and inducible promoters, strong promoters (e.g., T7, CMV, and the like) find use in the constructs described herein, particularly where high expression levels are desired in an in vivo (cell-based) or in an in vitro expression system. Other exemplary promoters include mouse mammary tumor virus (MMTV) promoters, Rous sarcoma virus (RSV) promoters, adenovirus promoters, the promoter from the immediate early gene of human CMV, and the promoter from the long terminal repeat (LTR) of RSV. Alternatively, the promoter can also be provided by, for example, a 5′UTR of a retrovirus.

In preferred embodiments, mutated JAK2 is under the control of an inducible promoter, such as tetracycline-controlled transcriptional activation where transcription is reversibly turned on (Tet-On) or off (Tet-Off) in the presence of the antibiotic tetracycline or a derivative thereof, such as doxycycline. In a Tet-Off system, expression of tetracycline response element-controlled genes can be repressed by tetracycline and its derivatives. Tetracycline binds the tetracycline transactivator protein, rendering it incapable of binding to the tetracycline response element sequences, preventing transactivation of tetracycline response element-controlled genes. In a Tet-On system on the other hand, the tetracycline transactivator protein is capable of initiating expression only if bound by tetracycline; thus, introduction of tetracycline or doxycycline initiates the transcription of mutated JAK2. Another inducible promoter system known in the art is the estrogen receptor conditional gene expression system. Compared to the Tet system, the estrogen receptor system is not as tightly controlled; however, because the Tet system depends on transcription and subsequent translation of a target gene, the Tet system is not as fast-acting as the estrogen receptor system.

In general, the inducible promoters of use in the present invention are not particularly limited, as long as the promoter is capable of inducing expression of the downstream gene in response to an external stimulus. An example of such a promoter includes: a promoter capable of inducing expression of the downstream gene by binding to a complex including a tetracycline antibiotic (tetracycline, doxycycline, or the like) and a tetracycline transactivator in a case where the external stimulus is the presence of the tetracycline antibiotic; a promoter capable of inducing expression of the downstream gene by release of a tetracycline repressor in a case where the external stimulus is the absence of a tetracycline antibiotic; a promoter capable of inducing expression of the downstream gene by binding of an ecdysteroid (ecdysone, muristerone A, ponasterone A, or the like) to an ecdysone receptor-retinoid receptor complex in a case where the external stimulus is the presence of the ecdysteroid; and a promoter capable of inducing expression of the downstream gene by binding of FKCsA to a complex including a Gal4 DNA binding domain fused to FKBP12 and a VP16 activator domain fused to cyclophilin in a case where the external stimulus is the presence of FKCsA.

The expression cassette may comprise, as necessary, an enhancer, a silencer, a selection marker gene (for example, a drug resistance gene such as a neomycin resistance gene), an SV40 replication origin, and the like. Further, those skilled in the art could construct an expression cassette capable of inducing expression of JAK2 at a desired expression level by appropriately selecting a combination of known enhancers, silencers, selection marker genes, terminators, and so forth in consideration of the type of the promoter utilized and so on. In addition, as necessary, an expression cassette may also be introduced into the target cells that is capable of constantly expressing in the nucleus a factor (for example, tetracycline transactivator, a tetracycline repressor, an ecdysone receptor-retinoid receptor complex, a complex including a Gal4 DNA binding domain fused to FKBP12 and aVP16 activator domain fused to cyclophilin) for inducing expression of JAK2 in response to an external stimulus.

Expression vectors generally have convenient restriction sites located near the promoter sequence to provide for the insertion of nucleic acid sequences (such as, in the present invention, a mutated JAK2) encoding proteins of interest (such as mutant JAK2). A selectable marker operative in the expression host may be present to facilitate selection of cells containing the vector. In addition, the expression construct may include additional elements. For example, the expression vector may have one or two replication systems; thus allowing it to be maintained in different organisms, for example in mammalian cells for expression and in a prokaryotic host for cloning and amplification. In addition the expression construct may contain a selectable marker gene to allow the selection of transformed host cells. Selection genes are well known in the art and will vary with the host cell used.

In addition to vector-based delivery of mutated JAK2, it is also contemplated that erythroid progenitor cells, blood stem cells or human pluripotent stem cells of choice can be engineered to produce human synthetic chromosomes that express mutant JAK2. Fully-functional human synthetic chromosomes offer several advantages over viral-based delivery systems including increased payload size, the fact that extrachromosomal maintenance avoids host-cell disruption, and transcriptional silencing of introduced genes and possible immunological complications are avoided. Currently, there are several methods for engineering human synthetic chromosomes, including the “top down” method, the “bottom up” method, creating minichromosomes, and induced de novo chromosome generation. The “bottom up” approach of synthetic chromosome formation relies on cell-mediated de novo chromosome formation following transfection of a permissive cell line with cloned α-satellite sequences, which comprise typical host cell-appropriate centromeres and selectable marker gene(s), with or without telomeric and genomic DNA. (For protocols and a detailed description of these methods see, e.g., Harrington, et al., Nat. Genet., 15:345-55 (1997); Ikeno, et al., Nat. Biotechnol., 16:431-39 (1998); Masumoto, et al., Chromosoma, 107:406-16 (1998), Ebersole, et al., Hum. Mol. Gene., 9:1623-31 (2000); Henning, et al., PNAS USA, 96:592-97 (1999); Grimes, et al., EMBO Rep. 2:910-14 (2001); Mejia, et al., Genomics, 79:297-304 (2002); and Grimes, et al., Mol. Ther., 5:798-805 (2002).) The “top down” approach of producing synthetic chromosomes involves sequential rounds of random and/or targeted truncation of pre-existing chromosome arms to result in a pared down synthetic chromosome comprising a centromere, telomeres, and DNA replication origins. (For protocols and a detailed description of these methods see, e.g., Heller, et al., PNAS USA, 93:7125-30 (1996); Saffery, et al., PNAS USA, 98:5705-10 (2001); Choo, Trends Mol. Med., 7:235-37 (2001); Barnett, et al., Nuc. Ac. Res., 21:27-36 (1993); Farr, et al., PNAS USA, 88:7006-10 (1991); and Katoh, et al., Biochem. Biophys. Res. Commun., 321:280-90 (2004).) “Top down” synthetic chromosomes are constructed optimally to be devoid of naturally-occuring expressed genes and are engineered to contain DNA sequences that permit site-specific integration of target DNA sequences onto the truncated chromosome, mediated, e.g., by site-specific DNA integrases.

A third method of producing synthetic chromosomes known in the art is engineering of naturally occurring minichromosomes. This production method typically involves irradiation-induced fragmentation of a chromosome containing a functional, e.g., human neocentromere possessing centromere function yet lacking α-satellite DNA sequences and engineered to be devoid of non-essential DNA. (For protocols and a detailed description of these methods see, e.g., Auriche, et al., EMBO Rep. 2:102-07 (2001); Moralli, et al., Cytogenet. Cell Genet., 94:113-20 (2001); and Carine, et al., Somat. Cell Mol. Genet., 15:445-460 (1989).) As with other methods for generating synthetic chromosomes, engineered minichromosomes can be engineered to contain DNA sequences that permit site-specific integration of target DNA sequences. The fourth approach for production of synthetic chromosomes involves induced de novo chromosome generation by targeted amplification of specific chromosomal segments. This approach involves large-scale amplification of pericentromeric/ribosomal DNA regions situated on acrocentric chromosomes. The amplification is triggered by co-transfection of excess DNA specific to the pericentric region of chromosomes, such as ribosomal RNA, along with DNA sequences that allow for site-specific integration of target DNA sequences and also a drug selectable marker which integrates into the pericentric regions of the chromosomes. (For protocols and a detailed description of these methods see, e.g., Csonka, et al., J. Cell Sci 113:3207-16 (2002); Hadlaczky, et al., Curr. Opini. Mol. Ther., 3:125-32 (2001); and Lindenbaum and Perkins, et al., Nuc. Ac. Res., 32(21):e172 (2004).) During this process, targeting to the pericentric regions of acrocentric chromosomes with co-transfected DNA induces large-scale chromosomal DNA amplification, duplication/activation of centromere sequences, and subsequent breakage and resolution of dicentric chromosomes resulting in a “break-off” satellite DNA-based synthetic chromosome containing multiple site-specific integration sites.

Alternatively, mutated JAK2 can be inserted into an endogenous JAK2 chromosomal site by site-specific recombination. Site-specific recombination requires specialized recombinases to recognize specific recombination sites and catalyze recombination at these sites. A number of bacteriophage- and yeast-derived site-specific recombination systems, each comprising a recombinase and specific cognate sites, have been shown to work in eukaryotic cells for the purpose of DNA integration and are therefore applicable for use in engineering cells to express mutant JAK2. Such site-specific recombination systems include but are not limited to the bacteriophage P1 Cre/lox system, yeast FLP-FRT system, and the Dre system of the tyrosine family of site-specific recombinases. Such systems and methods of use are described, for example, in U.S. Pat. Nos. 7,422,889; 7,112,715; 6,956,146; 6,774,279; 5,677,177; 5,885,836; 5,654,182; and 4,959,317, which are incorporated herein by reference to teach methods for using such recombinases. Other systems of the tyrosine family such as bacteriophage lambda Int integrase, HK2022 integrase, and systems belonging to a separate serine family of recombinases such as bacteriophage phiC31, R4Tp901 integrases are known to work in mammalian cells are also applicable for use in the present invention.

The methods of the invention preferably utilize site-specific recombination sites that utilize the same recombinase, but which do not facilitate recombination between the sites. For example, a Lox P site and a mutated Lox P site can be integrated into the genome of a host, but introduction of Cre into the host will not facilitate recombination between the two sites; rather, the LoxP site will recombine with another LoxP site, and the mutated site will only recombine with another similarly-mutated LoxP site. Examples of such mutated recombination sites include those that contain a combination of inverted repeats or those that comprise recombination sites having mutant spacer sequences. For example, two classes of variant recombinase sites are available to engineer stable Cre-loxP integrative recombination. Both exploit sequence mutations in the Cre recognition sequence, either within the 8-bp spacer region or the 13-bp inverted repeats. Spacer mutants such as 1ox511, 1ox5171, 1ox2272, m2, m3, m7, and m11 recombine readily with themselves but have a markedly reduced rate of recombination with the wild-type site. This class of mutants has been exploited for DNA insertion by recombinase mediated cassette exchange using non-interacting Cre-Lox recombination sites and non-interacting FLP recombination sites (see, e.g., Baer and Bode, Curr. Opin. Biotechnol., 12:473-480 (2001); Albert, et al., Plant J., 7:649-659 (1995); Seibler and Bode, Biochemistry, 36:1740-1747 (1997); and Schlake and Bode, Biochemistry, 33:12746-12751 (1994)).

Inverted repeat mutants represent the second class of variant recombinase sites. For example, LoxP sites can contain altered bases in the left inverted repeat (LE mutant) or the right inverted repeat (RE mutant). An LE mutant, lox71, has 5 bp on the 5′ end of the left inverted repeat that is changed from the wild type sequence to TACCG (see Araki, et al, Nucleic Acids Res, 25:868-872 (1997)). Similarly, the RE mutant, lox66, has the five 3′-most bases changed to CGGTA. Inverted repeat mutants are used for integrating plasmid inserts into chromosomal DNA with the LE mutant designated as the “target” chromosomal loxP site into which the “donor” RE mutant recombines. Post-recombination, loxP sites are located in cis, flanking the inserted segment. The mechanism of recombination is such that post-recombination one loxP site is a double mutant (containing both the LE and RE inverted repeat mutations) and the other is wild type (see, Lee and Sadowski, Prog. Nucleic Acid Res. Mol. Biol., 80:1-42 (2005); and Lee and Sadowski, J. Mol. Biol., 326:397-412 (2003)). The double mutant is sufficiently different from the wild-type site that it is unrecognized by Cre recombinase and the inserted segment is not excised.

Introduction of the site-specific recombination sites may be achieved by conventional homologous recombination techniques. Such techniques are described in references such as e.g., Sambrook and Russell, Molecular cloning: a laboratory manual, 3rd ed. (2001, Cold Spring Harbor, N.Y.: Cold Spring Harbor Laboratory Press); Nagy, Manipulating the mouse embryo: a laboratory manual, 3rd ed. (2003, Cold Spring Harbor, N.Y.: Cold Spring Harbor Laboratory Press); and Miller, et al., Genetic Recombination: Nucleic acid, Homology (biology), Homologous recombination, Non-homologous end joining, DNA repair, Bacteria, Eukaryote, Meiosis, Adaptive immune system, V(D)J recombination (2009).

Specific recombination into the endogenous JAK2 locus can be facilitated using vectors designed for positive or negative selection as known in the art. In order to facilitate identification of cells that have undergone the replacement reaction, an appropriate genetic marker system may be employed and cells selected by, for example, use of a selection medium. However, in order to ensure that the genome sequence is substantially free of extraneous nucleic acid sequences at or adjacent to the two end points of the replacement interval, desirably the marker system/gene can be removed following selection of the cells containing the replaced nucleic acid.

In one preferred aspect of the methods of the present invention, cells in which the replacement of all or part of the endogenous JAK2 locus has taken place are negatively selected upon exposure to a toxin or drug. For example, cells that retain expression of HSV-TK can be selected through use of appropriate use of nucleoside analogues such as gancyclovir. In another aspect of the invention, a positive selection system that is used based on the use of two non-functional portions of a marker gene, such as HPRT, that are brought together through a recombination event. These two portions are brought into functional association upon a successful JAK2 replacement reaction being carried out wherein the functionally reconstituted marker gene is flanked on either side by further site-specific recombination sites (which are different to the site-specific recombination sites used for the replacement reaction), such that the marker gene can be excised from the genome if desired, using an appropriate site-specific recombinase. The recombinase may be provided to the target cell as a purified protein, or a construct transiently expressed within the cell in order to provide the recombinase activity.

The mutated JAK2 expression vector can be delivered to the cells to be engineered and/or produce a synthetic chromosome by any method known in the art. The terms transfection and transformation refer to the taking up of exogenous nucleic acid, e.g., an expression vector, by a host cell whether or not any coding sequences are, in fact, expressed. Numerous methods of transfection are known to the ordinarily skilled artisan, for example, by Agrobacterium-mediated transformation, protoplast transformation (including polyethylene glycol (PEG)-mediated transformation, electroporation, protoplast fusion, and microcell fusion), lipid-mediated delivery, liposomes, electroporation, sonoporation, microinjection, particle bombardment and silicon carbide whisker-mediated transformation and combinations thereof (see, e.g., Paszkowski, et al., EMBO J., 3:2717-2722 (1984); Potrykus, et al., Mol. Gen. Genet., 199:169-177 (1985); Reich, et al., Biotechnology, 4:1001-1004 (1986); Klein, et al., Nature, 327:70-73 (1987); U.S. Pat. No. 6,143,949; Paszkowski, et al., in Cell Culture and Somatic Cell Genetics of Plants, Vol. 6, Molecular Biology of Plant Nuclear Genes, (Schell and Vasil, eds., Academic Publishers 1989); and Frame, et al., Plant J., 6:941-948 (1994)); direct uptake using calcium phosphate (Wigler, et al., PNAS U.S.A., 76:1373-1376 (1979)); polyethylene glycol (PEG)-mediated DNA uptake; lipofection (see, e.g., Strauss, Meth. Mol. Biol., 54:307-327 (1996)); microcell fusion (Lambert, PNAS U.S.A., 88:5907-5911 (1991); U.S. Pat. No. 5,396,767; Sawford, et al., Somatic Cell Mol. Genet., 13:279-284 (1987); Dhar, et al., Somatic Cell Mol. Genet., 10:547-559 (1984); and McNeill-Killary, et al., Meth. Enzymol., 254:133-152 (1995)); lipid-mediated carrier systems (see, e.g., Teifel, et al., Biotechniques, 19:79-80 (1995); Albrecht, et al., Ann. Hematol., 72:73-79 (1996); Holmen, et al., In Vitro Cell Dev. Biol. Anim , 31:347-351 (1995); Remy, et al., Bioconjug. Chem., 5:647-654 (1994); Le Bolch, et al., Tetrahedron Lett., 36:6681-6684 (1995); and Loeffler, et al., Meth. Enzymol., 217:599-618 (1993)); or other suitable methods. Methods for production of synthetic chromosomes are described in U.S. application Ser. No. 09/815,979. Successful transfection is generally recognized by detection of the presence of mutated JAK2 within the transfected cell, such as, for example, any visualization of the heterologous nucleic acid, expression of a selectable marker or any indication of the operation of a vector within the host cell. For a description of delivery methods useful in practicing the present invention, see U.S. Pat. No. 5,011,776; U.S. Pat. No. 5,747,308; U.S. Pat. No. 4,966,843; U.S. Pat. No. 5,627,059; U.S. Pat. No. 5,681,713; Kim and Eberwine, Anal. Bioanal. Chem. 397(8): 3173-3178 (2010).

### Culturing the Modified Hematopoietic Stem Cells and Inducing Enucleation

The blood cell/progenitor cell differentiation induction culture protocols for culturing the human pluripotent stem cells, human blood stem cells or human erythroid progenitor cells used in the present invention will depend upon the cell used, how much differentiation is required, the selection methods to be employed, etc. Further, improved methods for culturing human pluripotent stem cells, human blood stem cells and human erythroid progenitor cells are being developed continually. The present invention is not dependent on any particular cell or any particular culture/differentiation methods. For general methods of blood cell/progenitor cell differentiation induction culture, see, e.g., Murphy, et al., U.S. Pub. No. 2014/0050711; Nakamura, et al., US Pub. No. 2014/0024118; Lu, et al., Blood, DOI 10.1182/bllod-2008-05-157198 (Aug. 19, 2008); Olivier, et al., Stem Cells Transl. Med., 1:604-14 (2012); and Hiroyama, et al., Stem Cells Int'l, DOI:10.4061/2011/195780 (2011), all of which are incorporated herein in their entirety.

The method for inducing differentiation of human pluripotent stem cells to human blood stem cells to erythroid cells is not limited. For example, first human pluripotent stem cells may be co-cultured with feeder cells in the presence of human insulin-like growth factor-II (IGF-II) and human vascular endothelial growth factor (VEGF) to induce differentiation into blood stem cells. Then, differentiation into a cell population including erythroid cells can be induced by co-culturing with feeder cells in the presence of human stem cell factor (SCF), human erythropoietin (EPO), and dexamethasone (DEX) in place of IGF-II and VEGF.

The human blood stem cells comprising the JAK2 expression cassette are cultured in the presence of an external stimulus and a blood growth factor. A “blood growth factor” means a factor contributing to the differentiation induction of blood stem cells into erythroid progenitor cells or to expansion of the erythroid progenitor cells. Examples of such “blood growth factors” include SCF, EPO, TPO (thrombopoietin), and DEX. A suitable concentration of SCF typically is from 50 to 100 ng/ml, a suitable concentration of EPO typically is 3 to 5 U/ml, a suitable concentration of TPO typically is 50 to 100 ng/ml, and suitable concentration of DEX typically is on the order of 10-6 M. Also, if a Tet-ON system is used, a suitable concentration of doxycycline typically is in the range of 1 to 2 μg/ml.

Examples of culture media useful for the methods of the present invention include Iscove's Modified Dulbecco's Medium (IMDM), α-Minimum Essential Medium (αMEM), and Dulbecco's Modified Eagle Medium (DMEM). Further, the culture medium in preferred methods contains additional factors such as fetal bovine serum (FBS), bovine serum albumin (BSA), human insulin, human transferrin, 2-mercaptoethanol, sodium selenate, ascorbic acid, alpha-monothioglycerol, L-glutamine, and the like. In addition, inorganic salts (such as ferrous sulfate), antibiotics (such as, for example, streptomycin, penicillin), and the like may be employed.

When a blood cell population (including blood stem cells derived from the human pluripotent stem cells or blood stem cells derived from human umbilical cord blood or the like) is induced to differentiate into the erythroid progenitor cells, it is suitable to use, e.g., IMDM medium containing FBS, human insulin, human transferrin, sodium selenite, ascorbic acid, alpha-monothioglycerol, L-glutamine, and the like. On the other hand, for cRBCs to be used in a clinical application, a serum-free culture medium may be preferred. For inducing differentiation into erythroid progenitor cells in a serum-free medium, IMDM containing human insulin, human transferrin (human iron-saturated (holo) transferrin) and 2-mercaptoethanol may be employed. An immortalized cell line is considered to be established after continuous culture of at least 3 months, and more preferably, an immortalized cell line is considered to be established when the culture is continued for at least 6 months.

As mentioned above, inducing differentiation of cells at different phases may require co-culture of the human pluripotent or blood stem cells with a feeder cell layer. Examples of feeder cells include OP9 cells, MEF, SNL76/7 cells, PA6 cells, NIH3T3 cells, M15 cells, 10T1/2 cells, and the like. These feeder cells are preferably used after exposed to radiation or treated with a cell division inhibitor (such as mitomycin C) to stop the cell division.

In vitro enucleation and separation of extruded nuclei from the modified, cultured RBCs can be achieved by various methods. For example, co-culturing maturing cRBCs with a stromal cell line such as a human mesenchymal cell line (see, e.g., Giarrtana, et al., Nat. Biotechnology, 23:69-74 (2005)) is known to drive enucleation. Alternatively, optimization of culture conditions using, e.g., IMDM, α-MEM, or DMEM comprising, e.g., human plasma protein fraction, human serum, D-mannitol, adenine, sodium hydrogen phosphate, mifepristone, α-tocopherol, linoleic acid, cholesterol, sodium selenite, human holo-transferrin, human insulin, ethanolamine, 2-mercaptoethanol with human stem cell factor (SCF), human erythropoietin (EPO), and interleukin 3 (or with VEGF and IGF-II) may promote culture and enucleation without co-culture with another cell line (see, e.g., Miharada, et al., Nat. Biotechnology, 24:1255-56 (2006); and Timmins, et al., Tissue Eng. Part C Methods, 17:1131-37 (2011)).

### Isolating the Mature Mutated Cultured RBCs

The RBCs may be enriched using any convenient method known in the art, including fluorescence activated cell sorting (FACS), magnetically activated cell sorting (MACS), density gradient centrifugation and the like. Parameters employed for enriching certain cells from a mixed population include, but are not limited to, physical parameters (e.g., size, shape, density, etc.) and molecule expression (e.g., expression of cell surface proteins or carbohydrates, reporter molecules, e.g., green fluorescent protein, etc.). Density gradient centrifugation is particularly cost effective, and can be used safely for mature RBCs in which no white blood cells are likely to be present, such as mature RBCs produced from an immortalized erythroid progenitor cell line. In alternative embodiments, an affinity purification method is utilized to isolate modified RBCs that have cell-surface antibodies that bind to a specific antigen, either naturally, or cRBCs that have been engineered to do so. The antigen used to immobilize the cRBCs may be immobilized on a solid phase and used to selectively retain the cRBCs, while other nucleated RBCs and other cells are washed away. The retained mature RBCs may then be eluted by a variety of methods, such as by chaotropic agents, changing the pH, salt concentration, etc. Any of the well-known methods for immobilizing or coupling an antigen to a solid phase may be used. In the instances where the antigen is a protein, the protein may be covalently attached to a solid phase, for example, sepharose beads, by well-known techniques, etc.

Alternatively, a labeled antigen may be used to specifically label cells that express an antibody that binds to the antigen and the labeled cells may then be isolated by cell sorting (e.g., by FACS). In certain cases, methods for antibody purification may be adapted to isolate antibody producing cells. Such methods are well known and are described in, for example, Sun, et al., J. Immunol. Methods, 282(1-2):45-52 (2003); Roque et al., J. Chromatogr A., 1160(1-2):44-55 (2007); and Huse, et al., J. Biochem. Biophys. Methods, 51 (3):217-31 (2002). The RBCs may also be isolated using magnetic beads or by any other affinity solid phase capture method, protocols for which are known. In some embodiments, antigen-specific antibody producing cells may be obtained by flow cytometry using the methods described in Wrammert, Nature, 453: 667-72 (2008), Scheid, Nature, 458: 636-40 (2009), Tiller, J. Immunol. Methods, 329 112-24 (2008); or Scheid, PNAS, 105: 9727-32 (2008), for example, all of which are incorporated by reference for disclosure of those methods. Exemplary antibody-producing cell enrichment methods include performing flow cytometry (FACS) of cRBC cell populations, e.g., through incubating the cells with labeled antigens and sorting the labeled cells using a FACSVantage SE cell sorter (Becton-Dickinson, San Jose, Calif.). Alternatively, mature enucleated modified cRBCs may be isolated from nucleated RBCs via optical red blood cell sorting via flow cytometry. Optical sorting offers an alternative to FACs or MACs, both of which require labelling of the cells (see, e.g., Ashkin, et al., Am. Soc. Of Graviational and Space Biol., 4(2):133-46 (1991)); and MacDonald, et al., Nature, 426:421-24 (2003)).

Further, the isolated red blood cells can be sterilized by irradiation. Because mature RBCs are enucleated, they remain functional after irradiation.

### Use of the Mature Mutated Cultured RBCs

A primary purpose for generating modified cRBCs is for blood transfusions in humans. As mentioned above, modified cRBCs have the advantages that reliance on the current volunteer-based collection system is not necessary, thus the supply of transfusable RBCs is not vulnerable to supply chain disruptions; the modified cRBCs of the present invention can be chosen to exhibit particular phenotypes such as being O/RhD-, and, further, with additional, more precise matching for chronically-infused patients; and the risk of contamination by pathogens is greatly reduced. Further, use of modified cRBCs has the additional advantage that the cells transfused are homogenous in age where the lifespan of the cells should be close to 120 days (the average lifespan of an RBC), as compared to the mean half-life of 28 days of RBCs collected from a donor, possibly reducing the number of transfusions and alleviating iron overload, a major complication in polytransfused patients. Thus, the modified cRBCs of the invention can be used in surgical and chemotherapy settings, and to treat blood diseases such as anemia, e.g., iron deficiency anemia, sickle-cell disease, thalassemia, spherocytosis syndromes, pernicious anemia, aplastic anemia; and hemolysis due to, e.g., malaria.

Yet another application of the modified cRBCs of the present invention is the production of reagent JAK2-modified cRBCs. Reagent JAK2-modified cRBCs are panels of cells with known antigen profiles that may be used prior to transfusion to test the serum of the recipient patient for the presence of antibodies that may react with the transfused cells. Current panels of reagent RBCs represent antigen profiles found primarily in Caucasian populations. Rare or uncommon phenotypes are essential for testing serum from patients that require frequent transfusions to help identify the great diversity of antibodies that develop when such patients become allo-immunized.

An additional use for the modified cRBCs of the present invention is as a research tool to screen drug candidates effective for treatment or prevention of blood-borne diseases, such as malaria. For example, the modified cRBCs of the present invention may be brought into contact with malarial parasites and a test compound to assess the efficacy of the compound to protect the RBCs from malarial infection.

It is also envisioned that the modified cRBCs of the present invention could be useful as a therapeutic vector. Because RBCs by nature have excellent biodistribution in the body and no longer divide, modified cRBCs could be engineered to produce cytoplasmic or membrane proteins with a deliberately limited duration of action for various therapeutic objectives.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventor to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments described herein. Rather, the scope and spirit of present invention is embodied by the appended claims. In the claims that follow, unless the term “means” is used, none of the features or elements recited therein should be construed as means-plus-function limitations pursuant to 35 U.S.C. § 112, ¶6.

I claim:

1. A method of producing a mature modified cultured red blood cell with a modified Janus kinase 2 V617F (mutant JAK2) locus comprising:
providing human pluripotent stem cells or human blood cells;
transforming the human pluripotent stem cells or human blood cells with an expression vector expressing mutant JAK2 or creating a V617F mutation in an endogenous JAK2 locus in the human pluripotent stem cells or human blood cells to produce JAK2 modified cells;
culturing the JAK2 modified cells;
inducing enucleation of the JAK2 modified cells; and
isolating the enucleated JAK2 modified cells.

2. The method of claim 1, wherein the human pluripotent stem cells or human blood cells are human embryonic stem cells, human embryonal carcinoma cells, human embryonic germ cells, human multipotent germline cells, human mesodermal stem cells, human mesenchymal stem cells, human induced pluripotent stem cells, or human erythroid progenitor cells.

3. The method of claim 2, wherein the human pluripotent stem cells or human blood cells are human embryonic stem cells.

4. The method of claim 2, wherein the human pluripotent stem cells or human blood cells are human induced pluripotent stem cells.

5. The method of claim 2, wherein the human pluripotent stem cells or human blood cells are erythroid progenitor cells.

6. The method of claim 2, wherein the human pluripotent stem cells or human blood cells are ABO type O and RhD negative.

7. The method of claim 1, wherein the human pluripotent stem cells or human blood cells are transformed with a mutant JAK2 expression vector.

8. The method of claim 7, where in the mutant JAK2 is under control of an inducible promoter.

9. The method of claim 1, wherein the human pluripotent stem cells or human blood cells comprise a human synthetic chromosome expressing mutant JAK2.

10. The method of claim 9, wherein the mutant JAK2 is under control of an inducible promoter.

11. The method of claim 1, wherein the endogenous JAK2 locus is replaced with mutant JAK2 via homologous recombination.

12. The method of claim 11, wherein the mutant JAK2 is under control of an inducible promoter.

13. The method of claim 1, wherein the modified cells are cultured in the presence of one or more of human insulin-like growth factor-II, human vascular endothelial growth factor, human stem cell factor, human erythropoietin or dexamethasone.

14. The method of claim 13, wherein the modified cells are cultured first in human insulin-like growth factor-II and human vascular endothelial growth factor, followed by culture in human stem cell factor, human erythropoietin and dexamethasone.

15. The method of claim 1, wherein the modified cells are cultured in the presence of feeder cells.

16. The method of claim 15, wherein the feeder cells are OP9 cells, MEF, SNL76/7 cells, PA6 cells, NIH3T3 cells, M15 cells, or 10T1/2 cells.

17. The method of claim 1, wherein enucleation is induced by culturing the modified cells with a human stromal cell line.

18. The method of claim 1, wherein enucleation is induced by one or more of human stem cell factor, human erythropoietin, human interleukin 3, human vascular endothelial growth factor or human insulin-like growth factor-II.

19. The mature, enucleated cultured red blood cells produced by the method of claim 1.

20. A method of treating a human patient comprising transfusing the patient with the mature, enucleated cultured red blood cell of claim 19.

21. The mature, enucleated cultured red blood cells of claim 19, wherein the mature, enucleated cultured red blood cells are ABO type O and RhD negative.

22. A method of producing an immortalized modified mutant Janus kinase 2 V617F (mutant JAK2) erythroid progenitor cell line comprising:
providing human pluripotent stem cells or human blood cells;
transforming the human pluripotent stem cells or human blood cells with an expression vector expressing V617F mutant JAK2 or creating the Janus kinase 2 V617F mutation in an endogenous JAK2 locus in the human pluripotent stem cells or human blood cells to produce modified cells; and
culturing the modified cells in nondifferentiating blood stem/blood progenitor cell culture medium.

23. A method for producing a mature Janus kinase 2 V617F (mutant JAK2) modified cultured red blood cell from the immortalized modified mutant Janus kinase 2 V617F (mutant JAK2) erythroid progenitor cell line of claim 22, further comprising the steps of inducing enucleation of the modified cells; and isolating the enucleated modified cells.

24. A method of producing a mature Janus kinase 2 V617F (mutant JAK2) modified cultured red blood cell comprising:
providing human pluripotent stem cells, blood stem cells or erythroid progenitor cells from an individual who has a mutation in an endogenous JAK2 locus;
culturing the cells;
inducing enucleation of the modified cells; and
isolating the enucleated modified cells.
